(19) United States
(12) Patent Application Publication
(10) Pub. No.: US 2012/0021435 A1
(43) Pub. Date: Jan. 26, 2012

(54) DETECTION OF LIVING, CIRCULATING, OR DISSEMINATED CELLS OR CELL CONSTITUENTS IN BLOOD OR BONE MARROW FOLLOWING FILTRATION OF BLOOD

(75) Inventors: Karsten Hiltawsky, Schwerte (DE); Evamaria Stütz, Munchen (DE)

(73) Assignee: SIEMENS AKTIENGESELLSCHAFT, Munich (DE)

(21) Appl. No.: 13/187,792

(22) Filed: Jul. 21, 2011

(30) Foreign Application Priority Data
Jul. 23, 2010 (DE) 10 2010 032 081.1

(51) Int. Cl. G01N 21/64; G01N 21/17; C12M 1/40; G01N 33/53; C12M 1/34

(52) U.S. Cl. 435/7.5; 435/7.1; 435/288.7

(57) ABSTRACT

A method is disclosed for detecting circulating cells in a body fluid sample. In at least one embodiment, the method includes: filtering the body fluid sample through a porous membrane; transferring the porous membrane with the cells located thereon as filter residue to a cell culture vessel, wherein one surface of the cell culture vessel is coated with a first antibody which is directed to a first cell-specific marker; incubating the porous membrane in the cell culture vessel with a cell culture medium, wherein cell-specific markers released by any cells present are bound by the first antibody to produce a bound first cell-specific marker on the surface coated with the first antibody; removing the porous membrane with the cells located thereon as filter residue from the cell culture vessel; detecting the bound first cell-specific marker on the surface coated with the first antibody.

### PRIORITY STATEMENT

The present application hereby claims priority under 35 U.S.C. §119 to German patent application number DE 10 2010 032 081.1 filed Jul. 23, 2010, the entire contents of which are hereby incorporated herein by reference.

### FIELD

At least one embodiment of the invention is in the field of in vitro diagnostics and relates generally to a method for detecting living, circulating, or disseminated cells from body fluids (e.g., blood, urine) or tissue samples (e.g., bone marrow) mixed with fluid. In one embodiment, the method according to the invention is used in particular for obtaining and for analyzing circulating tumor cells and is therefore preferably applicable to tumor diagnostics.

In at least one embodiment, the detection of cells or cell constituents from peripheral blood or bone marrow is enabled by way of a functional test, after the blood or bone marrow has been filtered by way of a specific filtration method. The cells may be, in particular, circulating tumor cells (CTCs), mesenchymal stem cells from peripheral blood or bacteria from blood or other body fluids and also disseminated tumor cells (DTCs) from bone marrow.

In principle, at least one embodiment of the method can, however, also be extended to detect bacteria in peripheral blood (sepsis detection) or other body fluids, particularly when an incubation phase to improve the detection limit should be necessary.

### BACKGROUND

The occurrence of CTCs in peripheral blood is an indication of a possible dispersion of cells of a solid tumor at a very early stage, in which it is not yet possible to detect metastasis using customary imaging test methods (Pantel et al., 2009). Therefore, both the detection and the characterization of CTCs in peripheral blood are promising possibilities for identifying systemic tumor cell dissemination very early and for utilizing CTCs as prognostic markers. As a result, prognoses might be made and continuous observation of systemic therapies might be carried out. In addition, the characterization and evaluation of CTCs might be used as a diagnostic instrument to select a suitable treatment for solid tumors.

Systematic discovery of these cells in early tumor stages is possible with methods known to date, but only with limited accuracy. A major challenge in testing blood samples is the low number of circulating tumor cells. A test method therefore has to be of a sensitivity such that one tumor cell is detected per milliliter of blood. At the same time, the method has to be very specific, because a milliliter of blood contains, inter alia, about ten million leukocytes, which in part have similar properties to circulating tumor cells with respect to, for example, size, nucleus, etc., and in part have similar surface properties to circulating tumor cells.

Since CTCs occur in peripheral blood in extremely low concentrations (a few cells per ml of blood, i.e., a few epithelial cells to ˜1×107 leukocytes and ˜5×109 erythrocytes per ml of blood; Paterlini-Brechot and Benali, 2007), it is necessary to concentrate the target cells and to remove as many interfering cells (e.g., erythrocytes) as possible. The physical behavior of epithelial cells in blood is similar to that of leukocytes, i.e., when fractionating whole blood, CTCs are found in the leukocyte fraction. To fractionate whole blood or when accumulating epithelial cells or depleting superfluous cells, use is made of various methods, of which a few shall be briefly mentioned (Pantel et al., 2009; Paterlini-Brechot and Benali, 2007):

Density-gradient centrifugation with Ficoll-Hypaque, wherein mononuclear cells are isolated from the interphase which forms, with or without preceding negative selection of hematopoietic cells by using antibodies to leukocytes and erythrocytes (RosetteSep®, StemCell Technologies).

Immunomagnetic separation: either by positive selection for epithelial cells using epithelial-specific antibodies or by negative selection to deplete leukocytes using leukocyte-specific antibodies.

Size-based CTC accumulation by membrane filtration, wherein the pore size of the membrane filter is chosen such that any cells smaller than leukocytes are washed through and any cells similar in size to or larger than leukocytes are collected on the membrane.

Many of the methods mentioned can already be purchased on the open market as kits or as products, and are used alone or in combination. All of the methods mentioned have advantages and disadvantages, which shall be briefly discussed here: immunomagnetic separation to isolate and accumulate CTCs is dependent on the abovementioned antibodies used, and this could produce a distorted result. Especially in the case of positive selection via epithelial-specific antibodies, false-negative results can occur, since it can happen that tumor cells no longer express the usual epithelial marker antigens (e.g., EpCAM, cytokeratins) and therefore evade accumulation by positive selection. Alternatively, false-positive results can occur, since benign nontumor cells which may be epithelial and which may likewise be present in blood under special circumstances are also detected. Detection methods for isolated CTCs are used not only for counting but also mainly for further characterization (description) thereof. They comprise both immunocytological methods, by which epithelial-specific proteins (e.g., cytokeratins) or tumor-specific proteins (e.g., Her-2 in the case of breast carcinoma cells) are detected, and methods at the molecular level, such as the detection of specific DNA or RNA species (Pantel et al., 2009; Fehm et al., 2008; Paterlini-Brechot and Benali, 2007). The number of CTCs may also be determined in this way.

In the case of membrane filtration, the problem of marker-specific accumulation is not applicable, since all cells of a similar size are collected quantitatively, unless membrane pores are blocked by cell aggregates and impair filtration. The detection methods comprise, like immunomagnetic separation, descriptive (characterizing) immunocytology and molecular biology methods. Backwashing of the cells in membrane filtration into another medium is extremely difficult. Kahn and coworkers describe a recovery rate for epithelial cells after backwashing from the membrane filter of 53-63% (Kahn et al., 2004). However, false-positive results can also occur here when detection is limited only to the epithelial origin of the cells. Epithelial, benign nontumor cells, which may likewise be present in blood under special circumstances, are likewise detected.

The only method for isolating CTCs from blood or bone marrow in which the target cells can subsequently be tested with respect to functionality, i.e., to test whether the isolated epithelial cells are actually viable, potentially metastasizing tumor cells, is currently isolation and accumulation by way of density-gradient centrifugation. After centrifugation has been carried out, the target cells, together with the remaining leukocytes, are isolated from the interphase. A higher recovery rate for the target cells is obtained by carrying out negative selection of the hematopoietic cells using antibodies to leukocytes and erythrocytes prior to centrifugation (RosetteSep®, StemCells Technologies). The epithelial cells are detected either by the descriptive methods already mentioned above, or a functional test is carried out, in which extracellular specific proteins secreted by living, functional tumor cells are detected. For this purpose, the EPISPOT (epithelial immunospot) method is used. The isolated cells are seeded in membrane-coated multititer plates and cultured under cell culture conditions. The secreted proteins are subsequently detected using ELISA or immunofluorescence. However, the use of preceding negative selection represents a considerable cost factor per detection. Also, density-gradient centrifugation with subsequent removal of the interphase can be automated only with great difficulty.

### SUMMARY

In at least one embodiment, the present invention enables a reliable, cost-effective method for detecting (living) cells in a sample, in particular tumor cells in a blood sample.

In one embodiment, a method for detecting cells in a body fluid sample is provided. The method comprises:

(a) filtering the body fluid sample through a porous membrane having a pore size from about 0.1 to about 200 μm;

(b) transferring the porous membrane with the cells located thereon as filter residue to a cell culture vessel, wherein one surface of the cell culture vessel is coated with a first capture antibody which is directed to a first cell-specific marker;

(c) incubating the porous membrane in the cell culture vessel with a cell culture medium, wherein cell-specific markers released by any cells present are bound by the first antibody to produce a bound first cell-specific marker on the surface coated with the first antibody;

(d) removing the porous membrane with the cells located thereon as filter residue from the cell culture vessel; and

(e) detecting the bound first cell-specific marker on the surface coated with the first antibody.

In some embodiments, the method further comprises, after filtration, detecting a second cell-specific marker on the membrane with the cells located thereon as filter residue.

In another embodiment, a kit is provided for carrying out the methods of the invention described herein. The kit comprises

a) a porous membrane whose pore size is chosen such that cells having a nucleus are retained, whereas erythrocytes and smaller solid constituents are not retained,

b) a cell culture vessel, wherein one surface of the cell culture vessel is coated with a first antibody which is directed to a first cell-specific marker,

c) a first detection antibody which is directed to the first cell-specific marker and binds to an epitope other than that for the first antibody,

d) a second detection antibody which is directed to a second cell-specific marker, for detecting tumor cells directly on the membrane.

### BRIEF DESCRIPTION OF THE DRAWINGS

FIG. 1 is a diagram of the membrane after filtration.

FIG. 2 is a diagram of the arrangement according to the invention of membrane and cell culture vessel, with an enlarged cutout, in a first functional state.

FIG. 3 is a diagram of the enlarged cutout from FIG. 2 in a second functional state.

FIG. 4 is a diagram of the detection of the first cell-specific marker according to a first embodiment.

FIG. 5 is a diagram of the detection of the first cell-specific marker according to a second embodiment.

FIG. 6 is a diagram of the detection of the second cell-specific marker on the membrane using a second detection antibody.

FIG. 7 is a diagram of the detection of the second detection antibody using a secondary antibody.

### DETAILED DESCRIPTION

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

An embodiment of the invention relates to a method for detecting cells in a sample, comprising the following steps:

a) filtering the body fluid sample through a porous membrane having a pore size of from 0.1 to 200 μm,

b) transferring the membrane with the cells located thereon as filter residue to a cell culture vessel, wherein one surface of the cell culture vessel is coated with a first antibody which is directed to a first cell-specific marker, and wherein preferably the side of the membrane with the cells located thereon as filter residue is facing the coated surface,

c) incubating the membrane in the cell culture vessel with a cell culture medium for a predetermined period, wherein cell-specific markers released by any cells present are bound by the first antibody,

d) removing the membrane with the cells located thereon as filter residue from the cell culture vessel,

e) detecting the bound cell-specific marker on the surface coated with the first antibody.

The membrane is, according to an embodiment of the invention, transferred to the cell culture vessel and, for example, placed onto the base surface coated with a first antibody. Preferably, the side of the membrane with the cells located thereon as filter residue is facing the coated surface, although it is also conceivable for the membrane with the cells located thereon as filter residue to be arranged in the cell culture vessel facing away from the coated surface, and so cell-specific markers released by any cells present diffuse through the membrane and are then bound to the coated surface.

Possible samples are any liquid sample which may contain cells, in particular body fluids, for example blood, blood fractions, urine, saliva, cerebrospinal fluid, lymph, lacrimal fluid, and in addition as well rinse liquids from tissue or body cavities, for example bronchial lavages. Tissue samples, biopsies, and smears can also be collected and suspended in a suitable liquid (e.g., buffer, cell culture medium) and tested using the method of an embodiment of the present invention (e.g., bone marrow). Another possibility as well is samples from cell cultures in which a certain cell type is to be detected in the sample volume. The method is in principal suitable for detecting both prokaryotic and eukaryotic cells.

Cell-specific markers are cell- or tissue-specific substances which can be detected by antibodies, in particular peptides, proteins, glycoproteins, or fragments thereof, whose detection in the sample indicates the presence of a certain cell type, for example the presence of tumor cells. For instance, epithelial markers (e.g., EpCAM) in a blood sample are not to be expected in healthy individuals and their presence is an indication of CTCs.

Compared to the known EPISPOT method (Alix-Panabières et al.), in which the cells are isolated by gradient centrifugation, the method according to an embodiment of the invention offers numerous advantages:

isolation of the cells by membrane filtration is faster and more reliable than gradient centrifugation;

the size and/or shape of the membrane can be chosen such that it, in size and/or shape, substantially matches the coated surface;

the cells adhere to the membrane after filtration, which for example can be carried out by suction with low pressure or in a centrifugation tube (e.g., Falcon™ from Becton Dickinson), and are available for further analyses;

directly placing the membrane on the antibody-coated surface makes it possible to obtain, after detection of the first (secreted) cell-specific marker, a signal distribution pattern which mirrors the distribution of the (living) tumor cells on the membrane. The information contained in this pattern can be used in any further analysis of the cells on the membrane that may be performed. In particular, it is possible as a result to distinguish between secreting (and thus living) tumor cells and nonsecreting (and thus potentially nonviable) tumor cells.

Thus, according to one aspect of an embodiment of the invention, the cells on the membrane are further analyzed. This can be a visual analysis, for example using light or fluorescence microscopy. The cells can, for this purpose, be stained on the filter membrane, for example using nuclear staining, specific stainings of living or dead cells, and the like.

According to one aspect of an embodiment of the invention, the method comprises the further step of:

f) after filtration, detecting a second cell-specific marker on the membrane with the cells located thereon as filter residue.

The membrane has, according to an embodiment of the invention, a pore size of from about 0.1 to about 200 μm. As a result, cells can be retained, whereas cell fragments, thrombocytes, and smaller solid constituents of the sample pass through the filter (the membrane).

Depending on the use, it is also possible to carry out in succession two to three filtrations using decreasing pore diameters, so that especially small constituents (e.g., bacteria) can be cleaned up better.

According to an example aspect of an embodiment of the invention, the membrane has a pore size of from about 2 to about 50 μm, more preferably from about 5 to about 20 μm, even more preferably from about 5 to about 10 μm.

Pore sizes of the size ranges about 2 to about 50 μm, about 5 to about 20 μm, and about 5 to about 10 μm offer the advantage that the cells are retained thereby, but in part remain adhering in the pores and thus adhere especially well to the membrane and are available for further analyses.

According to one aspect of an embodiment of the invention, the first cell-specific marker and/or the second cell-specific marker are detected by way of an immunoassay, i.e., using detection antibodies.

According to one aspect of an embodiment of the invention, the base surface of the cell culture vessel is coated with the first antibody, wherein, in step (b), the membrane is placed onto the base surface with the side with the cells located thereon as filter residue facing downward.

According to one aspect of an embodiment of the invention, the cell culture vessel has at least one spacing device, so that the membrane can be incubated at a distance of about 2 mm or less, preferably about 1 mm or about 0.1 mm or about 0.02 mm or less, from the surface coated with the first antibody. The spacing device makes it possible to choose the distance such that, firstly, the cells on the membrane are supplied with cell culture medium and, secondly, prior to diffusion of the first cell-specific marker its becoming bound by the first antibody is limited.

According to one aspect of an embodiment of the invention, the cell culture vessel has at least one retaining device, so that the membrane can be fixed in a predefined position in the cell culture vessel for the duration of the incubation in step (c).

According to one aspect of an embodiment of the invention, the first and/or the second cell-specific marker is a cell-specific marker chosen from list 1 or 2.

In at least one embodiment, the sample is a blood sample.

According to an example aspect of an embodiment of the invention, when using a blood sample, erythrocyte lysis (e.g., by hypotonic lysis) is carried out prior to filtration in order to remove interfering erythrocytes.

According to an example aspect of an embodiment of the invention, the method comprises the additional step of:

g) after filtration, staining cells on the membrane using a dye.

For this purpose, dyes can be chosen which stain cells or cell constituents and are known from cytology and histology. These can be live or dead dyes, dyes which specifically stain nuclei or other organelles or which specifically stain certain cell components, for example nucleic acids or proteins. Known cell dyes are, for example, trypan blue, DAPI, and the like.

The cells can also be analyzed by microscopy on the membrane, stained or unstained.

In addition, the cells can also be recollected in cell culture medium after filtration and cultured for further tests. For instance, detected tumor cells can be cultured and further tested in order to check the response to certain drugs (e.g., cytostatics).

An embodiment of the invention further relates to a kit for carrying out at least one embodiment of the method, comprising:

a) a porous membrane whose pore size is chosen such that cells having a nucleus are retained, whereas erythrocytes and smaller solid constituents are not retained,

b) a cell culture vessel, wherein one surface of the cell culture vessel is coated with a first antibody which is directed to a first cell-specific marker,

c) a first detection antibody which is directed to the first cell-specific marker and binds to an epitope other than that for the first antibody,

d) a second detection antibody which is directed to a second cell-specific marker, for detecting tumor cells directly on the membrane.

According to one aspect of an embodiment of the invention, a labeled secondary antibody is provided for detecting the first and/or the second detection antibody.

According to one aspect of an embodiment of the invention, the first and/or the second detection antibody are/is labeled.

According to one aspect of an embodiment of the invention, the first and/or the second detection antibody and/or any secondary antibody present are labeled with a fluorophore or an enzyme.

According to one aspect of an embodiment of the invention, the kit further comprises a dye for staining cells on the membrane.

List 1: Preferred Cell-Specific Markers:

Alpha-1-fetoprotein (AFP) in hepatocellular carcinoma and gonadal and extragonadal germ cell tumors

Bence Jones protein in multiple myeloma

Beta hCG (beta subunit of human chorionic gonadotropin) in germ cell tumors of the ovary and nonseminomatous tumors of the testes

CA 15-3 in breast cancer (mammary carcinoma) or ovarian cancer (ovarian carcinoma)

CA 19-9 and CA 50 in pancreatic cancer (pancreatic carcinoma)

CA-125 in ovarian cancer (ovarian carcinoma)

Calcitonin (human calcitonin, hCT) in medullary thyroid carcinoma

Carcinoembryonic antigen (CEA) in bowel cancer, pancreatic carcinoma and adenocarcinoma of the lungs

Cytokeratin 21 fragment (CYFRA 21-1) and serpin B4 (SCC) in all variants of lung cancer (bronchial carcinoma)

HER-2/neu

HPV antibodies or HPV antigens

Homovanillic acid in neuroblastoma

5-Hydroxyindoleacetic acid in carcinoids

Catecholamines, vanillylmandelic acid in pheochromocytoma

Lactate dehydrogenase (LDH) in germ cell tumors

Lactate dehydrogenase isoenzyme 1 (LDH-1) in germ cell tumors; routine determination is, however, not yet recommended in current guidelines

MAGE antigens

Metanephrines in pheochromocytoma

MUC1 in non-small cell lung cancer (NSCLC) or in mammary carcinoma

NSE in small cell lung cancer (SCLC), neuroblastoma and seminomatous germ cell tumors

Placental alkaline phosphatase (PLAP) in seminomatous germ cell tumors

PSA in prostate cancer (prostate carcinoma)

Thyroglobulin (Tg) at any concentration in papillary or follicular thyroid carcinoma

Thymidine kinase

Cytokeratins, for example cytokeratin 8, 18, 19

List 2: Additional Cell-Specific Markers

β2-Microglobulin (β2-M)

CA 54-9

CA 72-4

CA 195

Cancer-associated serum antigen (CASA)

C-Peptide

Cytokeratin

Gastrin

Glucagon

Glucose-6-phosphate isomerase (GPI)

Insulin

Neopterin

Nuclear matrix protein 22 (NMP 22)

Ostase

p53 autoantibody

Paraproteins

Prolactin (PRL)

Protein S-100

Serpin 34 (SCC)

Pregnancy-specific β1-glycoprotein (SP-1)

Tumor-associated glycoprotein 12 (TAG 12)

Thymidine kinase (TK)

Tissue polypeptide antigen (TPA)

Tissue polypeptide-specific antigen (TPS)

Tumor M2-PK

Vasoactive intestinal polypeptide (VIP)

Transketolase-like 1 protein (TKTL1)

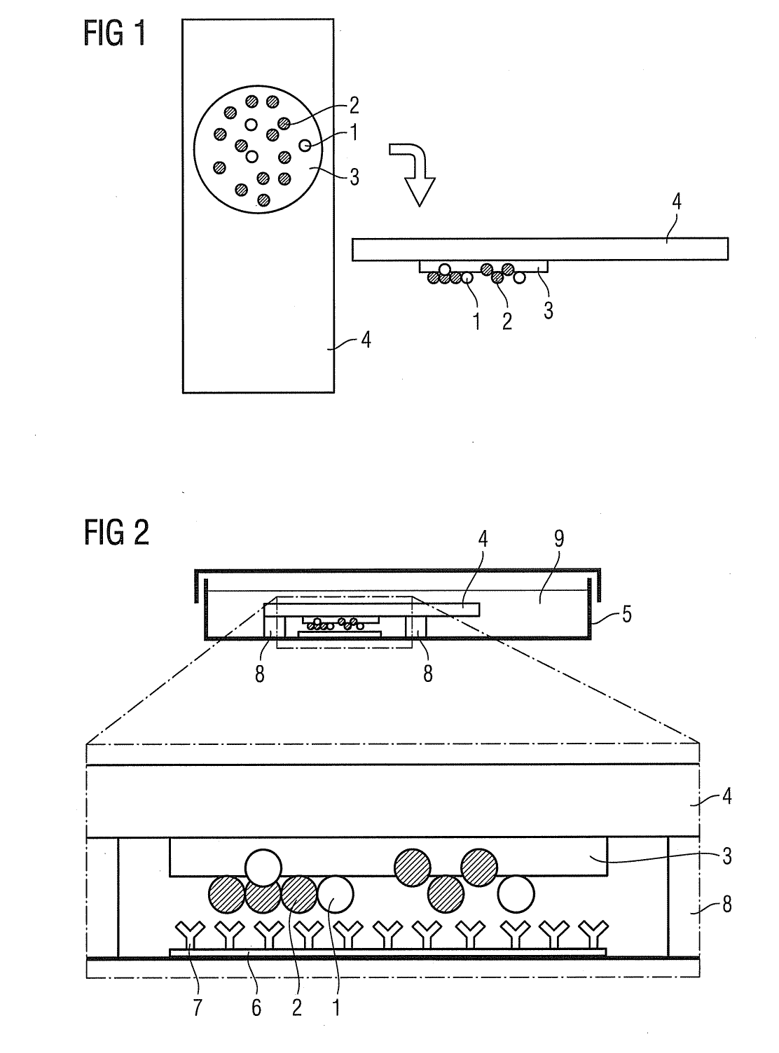

In FIG. 1, epithelial cells (1) are isolated from blood on the porous membrane (3) together with the leukocytes (2) by way of membrane filtration. For the filtration, use can be made of commercially available filters (e.g., track-etched filter membranes from Whatman). Suitable membrane materials are, for example, synthetic membranes (e.g., nylon, PE).

However, after filtration, no backwashing or other removal or transferring of the cells from the membrane (3) to another medium is carried out here. The membrane on which the cells have been collected is, along with the support (4) of the membrane, rotated by 180° with the cell-membrane side upside down (FIG. 1). In this orientation, it is placed into a vessel (5) (FIG. 2) whose base has been prepared as follows: on the surface (6) (plastic, glass, nitrocellulose, PVDF), there have been immobilized (covalently or by hydrophilic interactions) specific first capture antibodies (7) which are directed to cell-specific markers which are secreted by the cells, and so the cells which are trapped on the now bottom side of the filter membrane come into direct contact with the capture antibodies. In this way, the route which the secreted tumor-specific proteins travel via diffusion is limited to a minimum. The distance between the cells trapped on the membrane and the capture antibodies can be flexibly chosen by using at least one spacing device (8) variable in size, so as to ensure that the cells are sufficiently supplied with fresh cell culture medium (9), with which the cells are subsequently covered.

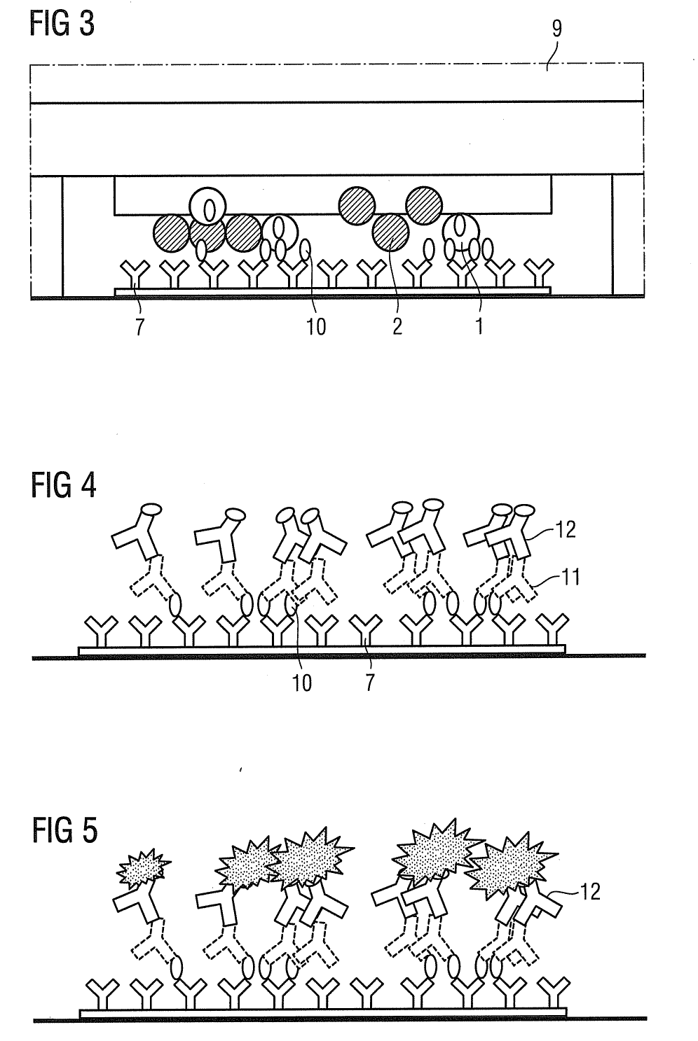

This arrangement is incubated under cell culture conditions (e.g., 37° C., in the incubator). During the incubation, in the case of isolated living and functional tumor cells, there is secreted a first cell-specific marker (10) which becomes bound by way of the base-immobilized first antibody (7) (FIG. 3). After a sufficient incubation time, the filter membrane with the cells located thereon is removed. The incubation time can be, for example, from 10 min to 1 h, 1 h to 24 h, 24 h to 48 h, or longer. Following this, two approaches are pursued further in the detection method:

1. the functional test, whether the epithelial cells are actually viable and, as potentially metastasizing cells, secrete specific tumor proteins into the extracellular medium (FIGS. 4 and 5, by way of example).

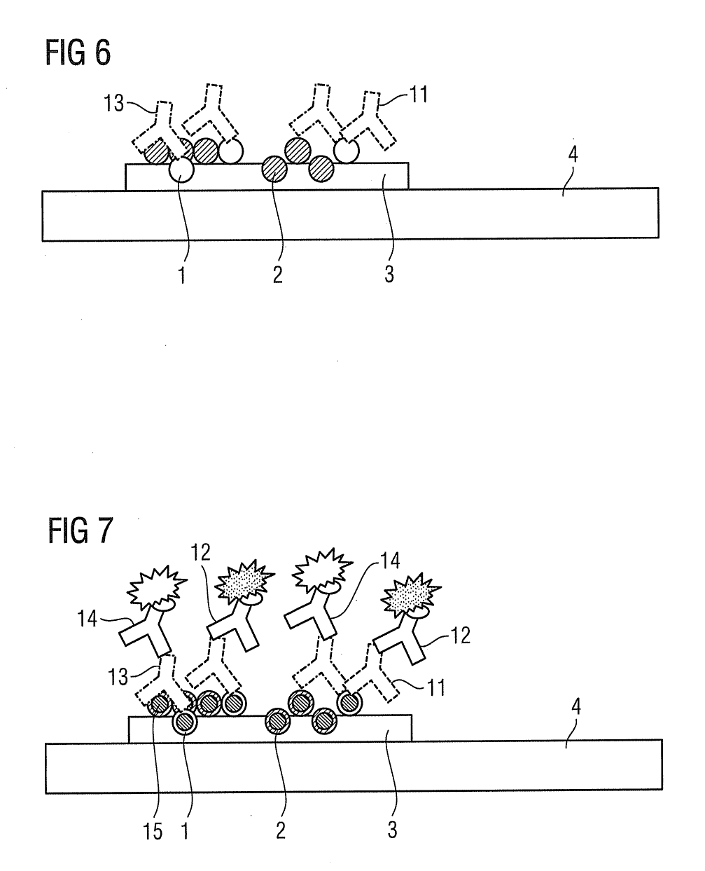

2. qualitative detection of the epithelial cells on the membrane (FIGS. 6 and 7, by way of example).

Functional Test:

In order to carry out the functional test, the cell culture medium is removed and the secreted proteins (10) bound to the capture antibodies are detected in the cell culture vessel via a subsequent ELISA or immunofluorescence (see figures). A primary detection antibody (11) binds to the tumor-specific secreted proteins immobilized by way of the capture antibodies (7).

When evaluating the signals by microscopy or in an automated manner in a scanner, spots are identified across the surface at those points over which originally the CTCs were located on the filter membrane. These spots produce a certain pattern.

Qualitative Detection of Epithelial Cells on the Filter Membrane:

The cells remaining on the filter membrane, in an unfixed (living) state or in a fixed (e.g., using paraformaldehyde) state, are stained by way of ELISA or immunofluorescence. As a second cell-specific marker, epithelial marker proteins, for example, can be detected (e.g., EpCAM, cytokeratins, . . . ) (11, 13), shown here for example as double staining. It is also possible to detect, for example, tumor-specific proteins (depending on the tumor type, for example HER-2, PSA, MUC-1, The various options for immunologically detecting proteins are known to a person skilled in the art. In the case of immunofluorescence, the nuclei can additionally be counterstained using a nuclear dye (15) (FIG. 7) to ensure better orientation of the cell distribution on the membrane. When evaluating the signals by microscopy or in an automated manner in a scanner, spots are identified across the surface at those points at which epithelial cells are located.

In the best case, there is obtained a negative image or a mirror-image pattern of the signals/spots compared to the pattern/signal distribution which was produced in the functional test for the secreted proteins. If the patterns of the two stains do not coincide, this positive-negative picture or this mirror-image picture enables epithelial living tumor cells to be distinguished from epithelial nonliving tumor cells, since preferably viable CTCs generate a signal in the functional test by secreting a protein. In addition, unspecific spots in the functional test can be classified as unspecific when no epithelial cell carrying a tumor-specific protein can be detected in the “membrane mirror image”.

Detection of antigen-antibody binding is possible in various ways:

The detection antibody (11, 13) is coupled to a fluorophore. The signal is read directly by way of fluorescence microscopy.

The detection antibody is coupled to an enzyme (e.g., HRP); after addition of a substrate, detection is carried out colorimetrically, i.e., after addition of a suitable substrate, a color change occurs as a result of the enzyme activity. The signal is read by way of, for example, light microscopy.

Detection is carried out by way of fluorescence, for example after addition of a fluorophore-coupled tyramide, the latter is activated by the appropriate enzyme (HRP). The highly reactive and transient tyramide resulting from the activation binds covalently to the proteins located in the immediate vicinity. Owing to this covalent binding, the fluorophore can be rendered visible in the immediate vicinity of the proteins to be detected (TSA; tyramide signal amplification; Invitrogen). The signal is read by way of fluorescence microscopy.

The detection antibody is coupled to biotin: after addition of streptavidin, to which a fluorophore is bound and which binds to biotin, the signal can be read directly by way of fluorescence microscopy.

After addition of streptavidin, to which a suitable enzyme (e.g., HRP, AP) is bound and which binds to biotin, there is added again a suitable substrate which is converted by the enzyme activity, leading to a color change. The signal is read by way of light microscopy.

After addition of streptavidin, to which a suitable enzyme (HRP) is bound and which binds to biotin, fluorophore-coupled tyramide is added again and activated by said suitable enzyme (HRP). The highly reactive and transient tyramide resulting from the activation binds covalently to the proteins located in the immediate vicinity. Owing to this covalent binding, the fluorophore can be rendered visible in the immediate vicinity of the proteins to be detected (TSA; tyramide signal amplification; Invitrogen). The signal is read by way of fluorescence microscopy.

The detection antibody is not coupled to anything and is detected using a specific secondary antibody (FIG. 7):

The secondary antibody (12) is coupled to, for example, a fluorophore (14). The signal is read directly by way of fluorescence microscopy (13).

The secondary antibody is coupled to a suitable enzyme (e.g., HRP): detection is carried out colorimetrically, i.e., after addition of a suitable substrate, a color change occurs as a result of the enzyme activity. The signal is read by way of light microscopy.

Detection is carried out by way of fluorescence, i.e., after addition of fluorophore-coupled tyramide, the latter is activated by the appropriate enzyme (HRP). The highly reactive and transient tyramide resulting from the activation binds covalently to proteins located in the immediate vicinity and can be rendered visible.

An embodiment of the method presented combines various known approaches for detecting scarce cells in blood or bone marrow, wherein in particular the advantages of the individual approaches are utilized and combined with one another:

By way of membrane filtration, all epithelial cells or tumor cells are quantitatively isolated from blood.

Loss of cells which occurs owing to possible backwashing steps of the filter membrane or the like is eliminated.

Nevertheless, the functionality of all the isolated cells can be tested without loss.

The cells are available for further analysis on the membrane after incubation, for example for cellular detection methods.

The mirror images of the results of the functional test and of the cellular detection are internal controls for false-positive or false-negative results.

Ease of automation

Owing to spacers, the cells which are located on the membrane and which are to be detected can both be completely surrounded by nutrient liquid and secrete proteins into the immediate surroundings of immobilized capture antibodies. Lastly, detection of the secreted proteins via immobilized capture antibodies forms the basis of the proposed functional test.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration, that is independent of the subject matters of the preceding dependent claims.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### Literature

Micrometastatic spread in breast cancer: detection, molecular characterization and clinical relevance, Tanja Fehm, Volkmar Müller, Catherine Alix-Panabieres and Klaus Pantel, Breast Cancer Research 2008, 10 (Suppl 1): S1

Enumeration of circulating tumor cells in the blood of breast cancer patients after filtration enrichment: correlation with disease stage, Harriette J. Kahn, Anthony Presta, Lu-Ying Yang, John Blondal, Maureen Trudeau, Lavina Lickley, Claire Holloway, David R. McCready, Daniel Maclean, and Alexander Marks, Breast Cancer Research and Treatment 2004, 86: 237-24

Cancer micrometastases, Klaus Pantel, Catherine Alix-Panabieres and Sabine Riethdorf, Nature Reviews in Clinical Oncology 2009, 6: 339-351

Circulating tumor cells (CTC) detection: Clinical impact and future directions, Patrizia Paterlini-Brechot and Naoual Linda Benali, Cancer Letters 2007, 253: 180-204

Full-length cytokeratin-19 is released by human tumor cells: a potential role in metastatic progression of breast cancer. Alix-Panabières C, Vendrell J P, Slijper M, Pellé O, Barbotte E, Mercier G, Jacot W, Fabbro M, Pantel K., Breast Cancer Res. 2009; 11 (3): R39. Epub 2009 Jun. 23

### LIST OF REFERENCE SYMBOLS

1 Epithelial cell
2 Leukocyte
3 Filter membrane
4 Filter membrane support
5 Cell culture vessel
6 Surface for antibody immobilization
7 Capture antibody (antibody directed to a tumor-specific, secreted protein, for example mouse anti-PSA)
8 Spacer
9 Cell culture medium
10 Tumor-specific, secreted protein (e.g., PSA)
11 Primary detection antibody (antibody directed to the same tumor-specific, secreted protein, but to an epitope other than that for the capture antibody, for example rabbit anti-PSA)
12 Secondary antibody with fluorophore (directed to detection antibody (11), for example anti-rabbit IgG-Alexa Fluor 488)
13 Detection antibody directed to a further epithelial marker protein (e.g., goat anti-CK8, preferably from a species other than that for the primary detection antibody 11)
14 Secondary antibody with fluorescent fluorophore (directed to detection antibody (13), for example anti-goat IgG-Alexa Fluor 546)
15 Nucleus, stained using nuclear dye (e.g., DAPI)

What is claimed is:

1. A method for detecting cells in a body fluid sample, comprising:
filtering the body fluid sample through a porous membrane having a pore size from about 0.1 to about 200 μm;
transferring the porous membrane with the cells located thereon as filter residue to a cell culture vessel, wherein one surface of the cell culture vessel is coated with a first capture antibody which is directed to a first cell-specific marker;
incubating the porous membrane in the cell culture vessel with a cell culture medium, wherein cell-specific markers released by any cells present are bound by the first antibody to produce a bound first cell-specific marker on the surface coated with the first antibody;
removing the porous membrane with the cells located thereon as filter residue from the cell culture vessel; and
detecting the bound first cell-specific marker on the surface coated with the first antibody.

2. The method as claimed in claim 1, further comprising:
after filtration, detecting a second cell-specific marker on the membrane with the cells located thereon as filter residue.

3. The method as claimed in claim 1, wherein the first cell-specific marker is detected by way of an immunoassay.

4. The method of claim 2, wherein the second cell-specific marker is detected by way of an immunoassay.

5. The method as claimed in claim 1, wherein the base surface of the cell culture vessel is coated with the first capture antibody and, in the transferring, the membrane is placed onto the base surface with the side of the membrane with the cells located thereon as filter residue facing downward.

6. The method as claimed in claim 1, wherein the cell culture vessel has at least one spacing device, so that the membrane can be incubated at a distance of 2 mm or less from the surface coated with the first antibody.

7. The method as claimed in claim 1, wherein the cell culture vessel has at least one retaining device, so that the membrane can be fixed in a predefined position in the cell culture vessel for the duration of the incubation.

8. The method as claimed in claim 2, wherein the first cell-specific marker and/or the second cell-specific marker comprises at least one marker selected from the group consisting of Alpha-1-fetoprotein (AFP), Bence Jones protein, Beta hCG, CA 15-3, CA 19-9, CA 50, CA-125, Calcitonin, Carcinoembryonic antigen (CEA), Cytokeratin 21 fragment (CYFRA 21-1), serpin B4 (SCC), HER-2/neu, an HPV antibody or HPV antigen, Homovanillic acid, 5-Hydroxyindoleacetic acid, a Catecholamine, vanillylmandelic acid, Lactate dehydrogenase (LDH), Lactate dehydrogenase isoenzyme 1 (LDH-1), a MAGE antigen, a Metanephrine, MUC1, NSE, Placental alkaline phosphatase (PLAP), PSA, Thyroglobulin (Tg), Thymidine kinase, a Cytokeratin, β2-Microglobulin (β2-M), CA 54-9, CA 72-4, CA 195, Cancer-associated serum antigen (CASA), C-Peptide, Cytokeratin, Gastrin, Glucagon, Glucose-6-phosphate isomerase (GPI), Insulin, Neopterin, Nuclear matrix protein 22 (NMP 22), Ostase, p53 autoantibody, a Paraprotein, Prolactin (PRL), Protein S-100, Pregnancy-specific β1-glycoprotein (SP-1), Tumor-associated glycoprotein 12 (TAG 12), Thymidine kinase (TK), Tissue polypeptide antigen (TPA), Tissue polypeptide-specific antigen (TPS), Tumor M2-PK, Vasoactive intestinal polypeptide (VIP) and Transketolase-like 1 protein (TKTL1)

9. The method as claimed in claim 1, wherein the cell is a tumor cell.

10. The method as claimed in claim 1, further comprising:
after filtration, staining cells on the membrane using a dye.

11. The method as claimed in claim 2, further comprising:
after filtration, staining cells on the membrane using a dye.

12. The method as claimed in claim 1, wherein the side of the membrane with the cells located thereon as filter residue is facing the coated surface.

13. A kit for carrying out the method of claim 1, comprising:
a porous membrane having a pore size of from about 0.1 to about 200 μm,
a cell culture vessel, wherein one surface of the cell culture vessel is coated with a first capture antibody which is directed to a first cell-specific marker, and
a first detection antibody which is directed to the first cell-specific marker and binds to an epitope other than that for the first antibody.

14. The kit as claimed in claim 13, further comprising:
a second detection antibody which is directed to a second cell-specific marker, for detecting cells on the membrane.

15. The kit as claimed in claim 14, wherein a labeled secondary antibody is provided for detecting the first and/or the second detection antibody.

16. The kit as claimed in claim 14, wherein at least one of the first and the second detection antibody is labeled.

17. The kit as claimed in claim 14, wherein the first and/or the second detection antibody and/or any secondary antibody present are labeled with a fluorophore or an enzyme.

18. The kit as claimed in claim 13, further comprising a dye for staining cells on the membrane.

19. The kit as claimed in claim 14, further comprising a dye for staining cells on the membrane.
